(19) United States
(12) Patent Application Publication
(10) Pub. No.: US 2009/0306653 A1
(43) Pub. Date: Dec. 10, 2009

(54) ELECTRODE SHEATH FOR A CATHETER

(75) Inventor: Neil Anderson, Roseville (AU)

Correspondence Address: MORRISON & FOERSTER LLP, 755 PAGE MILL RD, PALO ALTO, CA, 94304-1018, US

(73) Assignee: CathRx Ltd.

(21) Appl. No.: 12/282,292

(22) PCT Filed: Mar. 15, 2007
(86) PCT No.: PCT/AU07/00325

§ 371 Date: Jan. 21, 2009

Related U.S. Application Data
(60) Provisional application No. 60/783,320, filed on Mar. 16, 2006.

(51) Int. Cl. A61B 18/14

(52) U.S. Cl. 606/41

(57) ABSTRACT

An electrode sheath (10) for a catheter includes an elongate body member (12) having a proximal end and a distal end (14) and which defines a lumen (16) extending from the proximal end to the distal end (14). A part (18) of a periphery of the body member (12) is substantially flat to form a longitudinally extending planar receiving zone (20) for electrical conductors (32). An electrical conductor arrangement (22) is received in the receiving zone (20) and extends towards the distal end (14) of the body member (12). At least one substantially planar electrode (24) is arranged distally on the body member (12), the at least one electrode (24) overlying a distal region of the electrical conductor arrangement (22) and being in electrical contact with at least some of the conductors (32) of the electrical conductor arrangement (22).

### CROSS-REFERENCE TO RELATED APPLICATIONS

The present application claims priority from United States of America Provisional Patent Application No. 60/783,320 filed on 16 Mar. 2006, the contents of which are incorporated herein by reference.

### FIELD

This invention relates, generally, to catheters and, more particularly, to an electrode sheath for a catheter and to a catheter including such an electrode sheath.

### BACKGROUND

In the field of heat treatment of tissue, it is desirable if the device heating the tissue is in contact only with the tissue being treated and not surrounding tissue or bodily fluids. This minimises the power required to heat the tissue and also minimises unnecessary damage to other tissue, structures or fluid.

In addition, it is often necessary to overcome tissue irregularities at a site in a patient's body being heat treated. An example where a site in a patient's body is subjected to heat treatment is in the treatment of heart arrhythmias where tissue is ablated in an effort to cure the arrhythmia. The tissue is ablated to create a lesion to block the electrical impulses causing the arrhythmia. To ensure that a lesion of adequate depth is formed, it is desirable that the ablating electrode make good contact with the tissue. Other examples of the use of heat treatment at a site in a patient's body include treatment of Parkinson's disease, tumour ablation, endometriosis and pain management.

There is therefore a need for an electrode sheath for a catheter which meets these needs.

### SUMMARY

According to a first aspect of the invention, there is provided an electrode sheath for a catheter, the electrode sheath including

an elongate body member having a proximal end and a distal end and which defines a lumen extending from the proximal end to the distal end;

a part of a periphery of the body member being substantially flat to form a longitudinally extending planar receiving zone for electrical conductors;

an electrical conductor arrangement received by the receiving zone and extending towards the distal end of the body member; and

at least one substantially planar electrode arranged distally on the body member, the at least one electrode overlying a distal region of the electrical conductor arrangement and being in electrical contact with at least some of the conductors of the electrical conductor arrangement.

The electrode sheath may include a plurality of planar electrodes arranged at longitudinally spaced intervals along the body member, each electrode having a group of conductors of the conductor arrangement associated with it. The groups of conductors may terminate in staggered relationship relative to one another along the body member, an electrode being associated with, and overlying, each termination.

The body member may have a substantially D-shape when viewed in cross-sectional profile. Further, because the conductor arrangement is carried externally of the lumen, the lumen is unimpeded and a steering shaft is able to be received in the lumen. The lumen may also be used for the passage of irrigation fluid. Instead, the body member may define at least one secondary passage through which irrigation fluid can pass towards the electrodes.

The at least one secondary passage may be in communication with each electrode via at least one irrigation port associated with that electrode. It will be appreciated that each port may be arranged distally of its associated electrode or may open out into the electrode.

The electrical conductor arrangement may comprise a flat ribbon cable made up of side-by-side arranged, longitudinally extending insulated, conductors. The ribbon cable may be arranged on the planar portion of the body member and may be covered by a layer of insulating material. The insulation of the relevant conductors and a part of the insulation layer may be removed in use at the area where an electrode is to be formed to establish electrical contact between the conductors and the electrode.

In another embodiment, instead of, or in addition to, the secondary passages, a plurality of fluid carrying conduits may be interposed between the conductors of the ribbon cable for conveying irrigation fluid to the electrodes.

The body member may define a longitudinally extending lip along each side of the planar receiving zone with the ribbon cable being received and contained between the lips to inhibit snaring of the ribbon cable as the electrode sheath is negotiated through the vascular system of a patient, in use.

The at least one electrode may be a coating of a biocompatible material applied over the electrical conductor arrangement. Thus, the, or each, electrode may be flexible to enhance electrode-tissue contact, in use.

According to a second aspect of the invention, there is provided a component for an electrode sheath for a catheter, the component comprising

an elongate body member having a proximal end and a distal end and which defines a lumen extending from the proximal end to the distal end;

a part of a periphery of the body member being substantially flat to form a longitudinally extending planar receiving zone for electrical conductors; and

at least one secondary passage extending longitudinally through the body member.

A plurality of bores, or irrigation ports, may be arranged in longitudinally spaced relationship, the bores extending from at least one of the lumen and the secondary passage and opening out into the planar receiving zone of the body member.

The body member may define a longitudinally extending lip along each side of the planar receiving zone with an electrical conductor arrangement being receivable between the lips.

The body member may be of a flexible material. More particularly, the body member may be of a biocompatible plastics material such as a polyether block amide (PEBAX) or an elastomeric material such as a silicone material.

According to a third aspect of the invention, there is provided a component for an electrode sheath for a catheter, the component comprising

an elongate body member having a proximal end and a distal end and which defines a lumen extending from the proximal end to the distal end;

a part of a periphery of the body member being substantially flat to form a longitudinally extending planar receiving zone for electrical conductors; and

the body member defining a longitudinally extending lip along each side of the planar receiving zone, the lips being dimensioned to receive an electrical conductor arrangement between them, in use.

The invention extends also to a catheter which includes

an electrode sheath, as described above;

a handle to which the electrode sheath is mounted; and

a steering shaft extending from the handle and received in the lumen of the electrode sheath.

The electrode sheath and the steering shaft may be removably mounted on the handle.

### BRIEF DESCRIPTION OF DRAWINGS

FIG. 1 shows a plan view of a distal part of an electrode sheath, in accordance with an embodiment of the invention, for a catheter;

FIG. 2 shows an end view of the electrode sheath;

FIG. 3 shows an isometric view of a central part of the electrode sheath from one side;

FIG. 4 shows an isometric view of the part marked ‘A’ in FIG. 3;

FIG. 5 shows an isometric view of the central part of the electrode sheath from the other side; and

FIG. 6 shows a side view of a catheter in accordance with another embodiment of the invention.

### DETAILED DESCRIPTION OF EXEMPLARY EMBODIMENTS

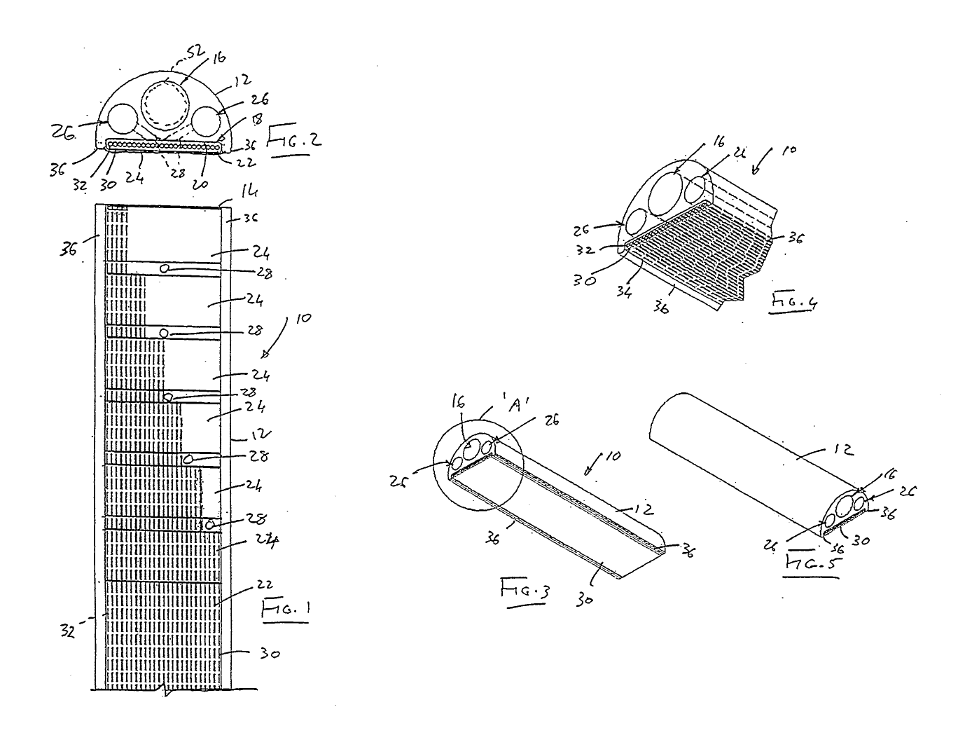

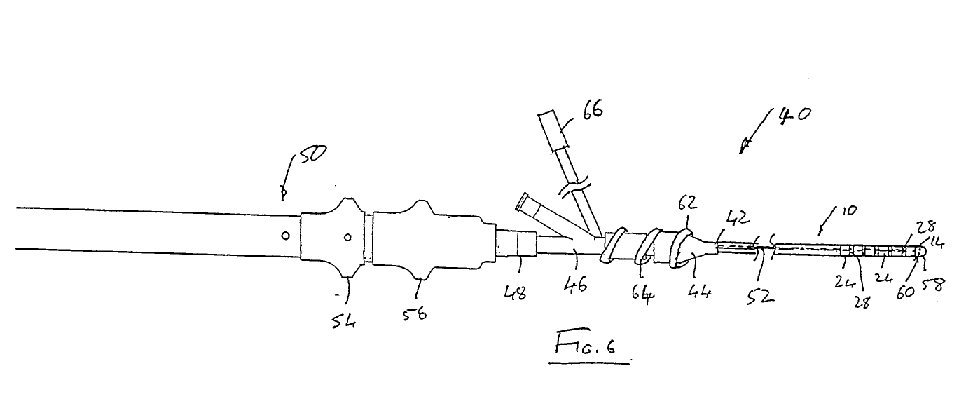

Referring initially to FIGS. 1 to 5 of the drawings, reference numeral 10 generally designates an electrode sheath, in accordance with an embodiment of the invention, for a catheter. The electrode sheath 10 comprises an elongate body member 12. The body member 12 has a proximal end (not shown in FIGS. 1-5) and a distal end 14. A lumen 16 is defined in the body member 12 extending from the proximal end to the distal end 14.

A part 18 of a periphery of the body member 12 is substantially flat to form a longitudinally extending planar receiving zone 20. An electrical conductor arrangement 22 is received in the receiving zone 20 extending towards the distal end 14 of the elongate body 12.

A plurality of substantially planar electrodes 24 are arranged at longitudinally spaced intervals on the electrical conductor arrangement 22 at a distal region of the elongate body 12 of the electrode sheath 10.

It will therefore be noted that, viewed end-on, the body member 12 has a substantially D-shape to provide the flat part 18 of the periphery of the elongate body 12.

In an embodiment, the elongate body 12 defines a plurality of secondary passages 26. The secondary passages 26 are used for conveying irrigation fluid from an irrigation source (not shown) to the electrodes 24.

The secondary passages 26 communicate with their associated electrodes 24 via irrigation ports 28. In the embodiment illustrated, the irrigation ports 28 open out distally of their associated electrodes. In respect of the most distal electrode 24, the irrigation fluid is ejected through the distal end 14 of the body member 12. It will be appreciated that the irrigation ports 28 could be arranged in other configurations relative to their associated electrodes 24. For example, the irrigation ports 28 could open out into their associated electrodes 24.

Instead of the secondary passages 26, or in addition thereto, the central lumen 16 may be shaped to permit the passage of irrigation fluid while still accommodating a steering shaft of a catheter of which the electrode sheath 10 forms a part. Thus, for example, the lumen 16 may be oval in cross-section.

The electrical conductor arrangement 22 is in the form of a flat ribbon cable 30. The ribbon cable 30 is formed by coating insulated conductors 32 with a layer 34 of an insulating material such as, for example, a polyether block amide (PEBAX). The conductors 32 have a diameter of about 0.1 mm and the layer 34 is about 0.05 mm thick. The ribbon cable 30 is bonded into the receiving zone 20 of the body member 12.

The conductors 32 of the ribbon cable 30 are arranged in groups. In the embodiment illustrated, six electrodes 24 are shown on the electrode sheath 10. In this embodiment, each electrode 24 has four conductors 32 associated with it, the four conductors defining a group. Two of the conductors 32 of the group are a copper pair for providing RF energy to the electrode 24. The other two conductors 32 of the group are a copper/constantan pair which act as a thermocouple for temperature sensing purposes associated with the relevant electrode 24.

The body member 12 defines a pair of opposed, longitudinally extending ribs or lips 36. These lips 36 define the recessed receiving zone 20 in which the ribbon cable 30 is received to lie substantially flush with an upper surface of the lips 36.

In another embodiment, the ribbon cable 30 may have a plurality of conduits interposed between the conductors 32. These conduits are used for conveying irrigation fluid to an associated electrode 24 in addition to, or instead of, the secondary passages 26.

The electrodes 24 are applied to the ribbon cable 30 in electrical contact with the relevant conductors 32 of the cable 30 using the techniques described in the Applicant's International Patent Application Nos. PCT/AU01/01339 dated 19 Oct. 2201 entitled “An electrical lead” and PCT/AU2005/000834 dated 10 Jun. 2005 entitled “A process of manufacturing an electrical lead”. The contents of these two patent applications are incorporated in this specification by reference. Instead, each electrode 24 may be in the form of a biocompatible metal foil, such as of Pt—Ir, and is applied to exposed regions of the relevant conductors 32 of the ribbon cable 30 using, for example, a conductive adhesive or by spot welding.

The elongate body 12 is of a suitable, flexible material such as, for example, polyether block amide (PEBAX) or an elastomeric material such as a silicone material. It will also be appreciated that, with the techniques used for forming the electrodes 24, the electrodes 24 are flexible. Thus, enhanced tissue-electrode contact is possible.

In FIG. 6 of the drawings, reference numeral 40 generally designates a catheter, in accordance with an embodiment of the invention. The catheter 40 includes the electrode sheath 10. A proximal end 42 of the electrode sheath 10 is secured to a mount 44. An irrigation supply connector 46 is connected to a proximal end of the mount 44. A proximal end of the connector 46 is connected to a distal end 48 of a catheter handle 50.

A steering shaft 52 extends from the handle into the lumen 16 of the electrode sheath 10. The steering shaft 52 is shown in FIG. 2 of the drawings on an enlarged scale.

The handle 50 includes a steering shaft controller 54 and a displacement mechanism 56. The steering shaft controller 54 is used to achieve steering of the electrode sheath 10 through the vascular system of a patient undergoing treatment. The displacement device 56 displaces the electrode sheath 10 relative to the steering shaft 52 so that a distal region of the electrode sheath 10 is not supported by the steering shaft 52. This allows greater flexibility of the distal region of the electrode sheath 10.

In the illustrated embodiment, a dome electrode 58 is mounted to the distal end 14 of the electrode sheath 10. As illustrated, the dome electrode 58 has apertures 60 defined therein through which irrigation fluid can be discharged.

A proximal end of the ribbon cable 30 is gathered into a telephone cable-like cord 62 having a coiled format. Coils 64 of the ribbon cable 30 are wound about the mount 44 and at least a distal portion of the irrigation supply connector 46 and terminate in a connector 66. The connector 66 connects to a source of ablation energy, such as radiofrequency (RF) energy (not shown).

It is a particular advantage of the invention that an electrode sheath 10 is provided which has flat electrodes 24. The flat electrodes 24 provide a greater surface area to be placed in contact with tissue to be treated. Further, because the electrodes 24 are not in contact with surrounding tissue, bodily structures or bodily fluids, the use of the flat electrodes 24 concentrates the energy on the site being treated. This has the benefit of affording finer control of the heat treatment and reduced energy consumption. Also, the use of the flat electrodes 24 enhances electrode-tissue contact. Due to the flexibility of the sheath 10 and the electrodes 24 themselves, this electrode-tissue contact is further enhanced.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An electrode sheath for a catheter, the electrode sheath including
an elongate body member having a proximal end and a distal end and which defines a lumen extending from the proximal end to the distal end;
a part of a periphery of the body member being substantially flat to form a longitudinally extending planar receiving zone for electrical conductors;
an electrical conductor arrangement received by the receiving zone and extending towards the distal end of the body member; and
at least one substantially planar electrode arranged distally on the body member, the at least one electrode overlying a distal region of the electrical conductor arrangement and being in electrical contact with at least some of the conductors of the electrical conductor arrangement.

2. The electrode sheath of claim 1 which includes a plurality of planar electrodes arranged at longitudinally spaced intervals along the body member, each electrode having a group of conductors of the conductor arrangement associated with it.

3. The electrode sheath of claim 2 in which the groups of conductors terminate in staggered relationship relative to one another along the body member, an electrode being associated with, and overlying, each termination.

4. The electrode sheath of claim 3 in which the body member defines at least one secondary passage through which irrigation fluid can pass towards the electrodes.

5. The electrode sheath of claim 4 in which the at least one secondary passage is in communication with each electrode via at least one irrigation port associated with that electrode.

6. The electrode sheath of claim 3 in which the electrical conductor arrangement comprises a flat ribbon cable made up of side-by-side arranged, longitudinally extending insulated, conductors.

7. The electrode sheath of claim 6 in which a plurality of fluid carrying conduits are interposed between the conductors of the ribbon cable for conveying irrigation fluid to the electrodes.

8. The electrode sheath of claim 1 in which the body member defines a longitudinally extending lip along each side of the planar receiving zone with the electrical conductor arrangement being received and contained between the lips.

9. The electrode sheath of claim 1 in which the at least one electrode is a coating of a biocompatible material deposited over the electrical conductor arrangement.

10. A component for an electrode sheath for a catheter, the component comprising
an elongate body member having a proximal end and a distal end and which defines a lumen extending from the proximal end to the distal end;
a part of a periphery of the body member being substantially flat to form a longitudinally extending planar receiving zone for electrical conductors; and
at least one secondary passage extending longitudinally through the body member.

11. The component of claim 10 in which a plurality of bores are arranged in longitudinally spaced relationship, the bores extending from at least one of the lumen and the secondary passage and opening out into the planar receiving zone of the body member.

12. The component of claim 10 in which the body member defines a longitudinally extending lip along each side of the planar receiving zone with an electrical conductor arrangement being receivable, in use, between the lips.

13. The component of claim 10 in which the body member is of a flexible material.

14. A component for an electrode sheath for a catheter, the component comprising an elongate body member having a proximal end and a distal end and which defines a lumen extending from the proximal end to the distal end;
a part of a periphery of the body member being substantially flat to form a longitudinally extending planar receiving zone for electrical conductors; and
the body member defining a longitudinally extending lip along each side of the planar receiving zone, the lips being dimensioned to receive an electrical conductor arrangement between them, in use.

15. The component of claim 14 in which at least one secondary passage extends longitudinally through the body member.

16. The component of claim 14 in which the body member is of a flexible material.

17. A catheter which includes
the electrode sheath of claim 1;
a handle to which the electrode sheath is mounted; and
a steering shaft extending from the handle and received in the lumen of the electrode sheath.

18. The catheter of claim 17 in which the electrode sheath and the steering shaft are removably mounted on the handle.
